SEQUENCE LISTING (25 sequences)

SEQ ID NO: 1 - DNA, 261 bp, Aspergillus niger
aagtcgtaacaaggtttccgtaggtgaacctgcggaaggatcattaccgagtgcgggtcctttgggcccaacctcccatccgtgtctattgtaccctgttgcttcggcgggcccgccgcttgtcggccgccgggggggcgcctctgccccccgggcccgtgcccgccggagaccccaacacgaacactgtctgaaagcgtgcagtctgagttgattgaatgcaatcagttaaaactttcaacaatggatctcttggttccg

SEQ ID NO: 2 - DNA, 235 bp, Stachybotrys echinata
aagtcgtaacaaggtctccgttggtgaaccagcggagggatcattaccgagtttacaactcccaaacccttatgtgaaccgtacctatcgttgcttcggcgggaacgccccggcgccctgcgcccggatccaggcgcccgccggagaccccaaactcttgtgtttttttcagtattctctgagtggcaaacgcaaaaataaatcaaaacttttaacaacggatctcttggctctg

SEQ ID NO: 3 - DNA, 252 bp, Penicillium
aagtcgtaacaaggtttccgtagggtgaacctgcggaaggatcattaccgagtgagggccctctgggtccaacctcccacccgtgtttattttaccttgttgcttcggcgggcccgccttaactggccgccggggggcttacgcccccgggcccgcgcccgccgaagacaccctcgaactctgtctgaagattgtagtctgagtgaaaatataaattatttaaaactttcaacaacggatctcttggttccg

SEQ ID NO: 4 - DNA, 271 bp, Alternaria soliaridae
aagtcgtaacaaggtctccgtaggtgaacctgcggagggatcattacacaatatgaaagcgggctggactccccccagcagtgcgttgctttgcggcgtgcgctgttggggccagccttgctgaattattcacccgtgtcttttgcgtacttcttgtttccttggtgggttcgcccaccacaaggacaaaccataaaccttttgtaattgcaatcagcgtcagtaacaataataataattacaactttcaacaacggatctcttggttctg

SEQ ID NO: 5 - DNA, 233 bp, Cladosporium halotolerans
aagtcgtaacaaggtctccgtaggtgaacctgcggagggatcattacaagttgaccccggcccccgggccgggatgttcacaaccctttgttgtccgactctgttgcctccggggcgaccctgcctccgggcgggggccccgggtggacacttcaaaactcttgcgtaactttgcagtctgagtaaatttaattaataaattaaaactttcaacaacggatctcttggttctg

SEQ ID NO: 6 - DNA, 264 bp, Neurospora terricola
aagtcgtaacaaggtctccgttggtgaaccagcggagggatcattacagagttgcaaaactccaacaaaccatcgcgaatcttacccgtacggttgcctcggcgctggcggtccggaaggccctcgggccccccggatcctcgggtctcccgctcgcgggaggctgcccgccggagtgccgaaaccaaactcttgatattttatgtctctctgagtaaacttttaaataagtcaaaactttcaacaacggatctcttggttctg

SEQ ID NO: 7 - DNA, 251 bp, Penicillium
aagtcgtaacaaggtttccgtaggtgaacctgcggaaggatcattaccgagtgagggccctctgggtccaacctcccacccgtgtttattttaccttgttgcttcggcgggcccgccttaactggccgccggggggcttacgcccccgggcccgcgcccgccgaagacaccctcgaactctgtctgaagattgtagtctgagtgaaaatataaattatttaaaactttcaacaacggatctcttggttccg

SEQ ID NO: 8 - DNA, 261 bp, Aspergillus piperis
aagtcgtaacaaggtttccgtaggtgaacctgcggaaggatcattaccgagtgcgggtcctttgggcccaacctcccatccgtgtctattataccctgttgcttcggcgggcccgccgcttgtcggccgccgggggggcgcctttgccccccgggcccgtgcccgccggagaccccaacacgaacactgtctgaaagcgtgcagtctgagttgattgaatgcaatcagttaaaactttcaacaatggatctcttggttccg

SEQ ID NO: 9 - DNA, 246 bp, Retroconis fusiformis
aagtcgtaacaaggtctccgttggtgaaccagcggagggatcattacagagttgcaaaactcccaaaccattgtgaacgttaccttcaaaccgttgcttcggcgggcggcccgggtccgcccggtgccccctggccccctcgcggggcgcccgccggaggaaacccaactcttgatacattatggcctctctgagtcttctgtactgaataagtcaaaactttcaacaacggatctcttggttctg

SEQ ID NO: 10 - DNA, 252 bp, Penicillium oxalicum
aagtcgtaacaaggtttccgtaggtgaacctgcggaaggatcattaccgagtgagggccctctgggtccaacctcccacccgtgtttatcgtaccttgttgcttcggcgggcccgcctcacggccgccggggggcatccgcccccgggcccgcgcccgccgaagacacacaaacgaactcttgtctgaagattgcagtctgagtacttgactaaatcagttaaaactttcaacaacggatctcttggttccg

SEQ ID NO: 11 - DNA, 240 bp, Preussia australis
tagaggaagtaaaagtcgtaacaaggtttccgtaggtgaacctgcggaaggatcattatcgtagggcttcggccctgtcgagatagaacccttgcctttttgagtaccttttcgtttcctcggcaggctcgcctgccaatggggaccccaaaaaacactttgcagtacctgtaaacagtctgaacaacctttaaaaattaaaactttcaacaacggatctcttggttctggcatcgatga

SEQ ID NO: 12 - DNA, 252 bp, Penicillium oxalicum
aagtcgtaacaaggtttccgtaggtgaacctgcggaaggatcattaccgagtgagggccctctgggtccaacctcccacccgtgtttatcgtaccttgttgcttcggcgggcccgcctcacggccgccggggggcatctgcccccgggcccgcgcccgccgaagacacacaaacgaactcttgtctgaagattgcagtctgagtacttgactaaatcagttaaaactttcaacaacggatctcttggttccg

SEQ ID NO: 13 - DNA, 284 bp, Mucor racemosus
aagtcgtaacaaggtttccgtaggtgaacctgcggaaggatcattaaataatcaataatcttggcttgtccattattatctatttactgtgaactgtattattatttgacgtttgagggatgttccaatgttataaggatagacattggagatgttaaccgagtcataatcaggtttaggcctggtatcctattattatttaccaaatgaattcagaattaatattgtaacatagacctaaaaaatctataaaacaacttttaacaacggatctcttggttctc

SEQ ID NO: 14 - DNA, 231 bp, Aspergillus subversicolor
aagtcgtaacaaggtttccgtaggtgaacctgcggaaggatcattaccgagtgcgggctgcctccgggcgcccaacctcccacccttgactacctaacactgttgcttcggcggggagccctctcgggggcgagccgccggggactactgaacttcatgcctgagagtgatgcagtctgagtctgaatataaaatcagtcaaaactttcaacaatggatctcttggttccg

SEQ ID NO: 15 - DNA, 240 bp, Phoma crystallifera
tagaggaagtaaaagtcgtaacaaggtttccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttctggcatcgatga

SEQ ID NO: 16 - DNA, 284 bp, Mucor circinelloides
aagtcgtaacaaggtttccgtaggtgaacctgcggaaggatcattaaataatcaataattttggcttgtccattattatctatttactgtgaactgtattattacttgacgcttgagggatgctccactgctataaggataggcgatggagatgctaaccgagtcataatcaagcttaggcttggtatcctattattatttaccaaaagaattcagaattaatattgtaacatagacctaaaaaatctataaaacaacttttaacaacggatctcttggttctc

SEQ ID NO: 17 - DNA, 287 bp, Malassezia restricta
aagtcgtaacaaggtttctgtaggtgaacctgcagaaggatcattagtgaagatttgggcaggccatacggacgccaaaaagtgtccctggccgcctacacccactatacatccacaaacccgtgtgcactgtcttggagaaaggcttcagagaagttttttgtggcctctcttggggtctttcttcgctacaaactcgaatggttagtatgaacgtggaacttggttggaccgtcactggccaacaaactatacacaactttcgacaacggatctcttggttctcc

SEQ ID NO: 18 - DNA, 405 bp, Cladosporium sphaerospermum
aagtcgtaacaaggtctccgtaggtgaacctgcggagggatcattaatcgacgaagtgcgtagctagacgcccggccgttttcgacccccggtaaccccggggggcggccgatcagcgtgctcagttaccaggccactcaccggagcgcgcccctgcgggggtagcgtggggaggggagagctcccgctaaggttgtagccgaccccgtttgtacctgcgcccgtgatggtcggatcttcatcaaaaccctttgttgtccgactctgttgcctcgggggcgaccctgcccttcattgggctcgggggacccccggtggacattaaccaaactcttgcgtatctttgtcgtctgagtgattttataaatcaaattaaaactttcaacaacggatctcttggttctg

SEQ ID NO: 19 - DNA, 240 bp, Phoma crystallifera
tagaggaagtaaaagtcgtaacaaggtttccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctctttgttctggcatcgatga

SEQ ID NO: 20 - DNA, 230 bp, Pestalotiopsis
aagtcgtaacaaggtctccgttggtgaaccagcggagggatcattacagagttatccaactcccaaacccatgtgaacttatctctttgttgcctcggcgcaagctacccgggacctcgcgccccgggcggcccgccggcggacaaaccaaaactcttgttatcttagttgattatctgagtgtcttatttaataagtcaaaactttcaacaacggatctcttggttctg

SEQ ID NO: 21 - DNA, 240 bp, Phoma crystallifera
tagaggaagtaaaagtcgtaacaaggtttccgtaggtgaacctgcggaaggatcattacctagagtttgtggacttcggtctgctacctcttacccatgtcttttgagtaccttcgtttcctcggcgggtccgcccgccggttggacaacattcaaaccctttgcagttgcaatcagcgtctgaaaaaacttaatagttacaactttcaacaacggatctcttggttcttgcatcgatga

SEQ ID NO: 22 - DNA, 323 bp, Ustilago striiformis
aagtcgtaacaaggtatctgtaggtgaacctgcagatggatcatttcgatgaaaaccttttttttcttgaggtgtggctcgcacctgtctaactaaacttgagctaccttttttcaacacggttgcatcggttggcctgtcaaacagtgcggcggcgtgaattttcacgtctgctttggctgggcgacggaccgacacttaatcaacacttttgatgatctaggatttgaatgataaaagttcatttttacaatgaaatcgactggtaatgcggtcgtctaatttttaaaaacaacttttggcaacggatctcttggttctcc

SEQ ID NO: 23 - DNA, 312 bp, Ustilago crameri
aagtcgtaacaaggtatctgtaggtgaacctgcagatggatcatttcgatgaaaaaccttttttttcgtgaggtgtggctcgcacctgtctaactaaaccgagctaccattttcaacacggttgcacggggtaggcctgtcagatagcgcgcgaattgattttcgaggctggacgaccgggtctaccatcaacattaaacactttttgatgatctaggatttgaaggaagttcattttacgatggaaccgactggtaatgcggtcgtctaaatctaaaaaataacttttggcaacggatctcttggttctcc

SEQ ID NO: 24 - DNA, 284 bp, Phanerochaete chrysorhiza
aagtcgtaacaaggtttccgtaggtgaacctgcggaaggatcattaacgagttttgaaatgggttgtagctggcctttgaaaaaatagaaggcatgtgcacgccctgctcatccactctcatacccctgtgcacttattgtaggcttgggtgggatgatcaactgtaaggttggtttgaaagcctttagtctatgctttattacaaactctacaaagtcatagaatgtcacattagcgtataacgcaataaatacaactttcagcaacggatctcttggctctc

SEQ ID NO: 25 - DNA, 251 bp, Penicillium aurantiogriseum
aagtcgtaacaaggtttccgtaggtgaacctgcggaaggatcattaccgagtgagggccctctgggtccaacctcccacccgtgtttattttaccttgttgcttcggcgggcccgccttaactggccgccggggggcttacgcccccgggcccgcgcccgccgaagacaccctcgaactctgtctgaagattgaagtctgagtgaaaatataaattatttaaaactttcaacaacggatctcttggttccg

(19) United States
(12) Patent Application Publication
(10) Pub. No.: US 2023/0038963 A1
(43) Pub. Date: Feb. 9, 2023

(54) COMPUTER-IMPLEMENTED METHODS OF IDENTIFYING MOLD GROWTH

(71) Applicant: YALE UNIVERSITY, New Haven, CT (US)

(72) Inventors: Jordan Peccia, Hamden, CT (US); Bridget Hegarty, Ann Arbor, MI (US)

(21) Appl. No.: 17/783,201

(22) PCT Filed: Dec. 11, 2020
(86) PCT No.: PCT/US2020/064669

§ 371 Date: Jun. 7, 2022

Related U.S. Application Data
(60) Provisional application No. 62/947,386, filed on Dec. 12, 2019.

(51) Int. Cl. G16B 30/00; G16B 40/20

(52) U.S. Cl. CPC G16B 30/00; G16B 40/20

(57) ABSTRACT

A computer-implemented method includes: receiving a set of DNA sequences extracted from one or more dust samples collected from a structure; analyzing the sequences using a machine learning estimator, where the machine learning estimator has been trained to distinguish structures with mold growth due to water damage from structures without mold growth due to water damage; and determining if the structure has mold growth due to water damage.

### CROSS-REFERENCE TO RELATED APPLICATION

This application claims the benefit of priority to U.S. Provisional Patent Application Ser. No. 62/947,386, filed Dec. 12, 2019. The entire content of this application is hereby incorporated by reference herein.

### STATEMENT REGARDING FEDERALLY SPONSORED RESEARCH OR DEVELOPMENT

This invention was made with government support under 1752134 awarded by National Science Foundation and under 14-2-1201497-94802 awarded by U.S. Department of Housing and Urban Development. The government has certain rights in the invention.

### BACKGROUND

Mold growth due to water damaged building materials is strongly associated with negative health impacts in building occupants. Mold and water damage in homes often occurs in hidden spaces (e.g., in walls, under floors, in ventilation systems) and/or in small amounts. When a mold inspector is called to a building, she is asked to make a determination if there are safe levels of mold in the building, if the building needs remediation, or, if remediation has occurred, if the building is free of mold. Insurance companies or prospective home buyers have similar questions. However, inspectors, restorers, or other interested parties do not have a simple, accurate test to determine the mold status of a building.

Guidelines for building inspectors specify that a home has been successfully remediated when it has been returned to “condition 1,” which is defined as “an indoor environment that may have settled spores, fungal fragments or traces of actual growth whose identity, location, and quantity are reflective of a normal fungal ecology for a similar indoor environment.” However, the guidelines fail to define “normal fungal ecology” in buildings. This reflects that a clear understanding of the fungal ecology of buildings without mold growth does not yet exist.

For this reason, individual building inspectors rely on methods, such as visual inspection, culturing, and microscopic assessment, that do not adequately represent the diversity of fungi found in buildings. More recent methods, such as ERMI (the Environmental Relative Moldiness Index), attempt to leverage techniques such as qPCR to better distinguish homes. While qPCR-based methods are useful when applied to the geographic region for which they were developed, they need to be modified to account for variable abundance of certain fungi between geographic regions. For example, ERMI is limited to a low number of qPCR assays (e.g., 36 different assays), which limits the mold identification possibilities of a given sample. Further, results of ERMI are scored on a basic, isolated score for a defined, static set of mold types, without the ability to detect patterns of different molds, confidence levels, and the like.

Although some mold inspectors have attempted to actively sample air to determine if a home has a mold problem, these methods offer only a short snapshot of a highly dynamic system and are of questionable utility. Additionally, these systems are costly and difficult to install. Other inspectors have attempted to use specific genera, such as Aspergillus/Penicillium, as indicators of mold damage in a building. However, Penicillium has been commonly found colonizing humans' skin and numerous other taxa have been associated with mold damage. All of the methods described above do not adequately account for the diversity of fungal communities in buildings, limiting their effectiveness in assessing whether a home needs remediation.

Current methods such as testing for the number of Aspergillus spores in an air sample are demonstrably inaccurate and/or highly subjective, depending on the judgment of the tester.

### SUMMARY

One aspect of the disclosure provides a computer-implemented method of identifying mold growth due to water damage in a structure. The computer-implemented method includes: receiving a set of DNA sequences extracted from one or more dust samples collected from the structure; analyzing the sequences using a machine learning estimator, where the machine learning estimator has been trained to distinguish structures with mold growth due to water damage from structures without mold growth due to water damage; and determining if the structure has mold growth due to water damage.

This aspect can include a variety of embodiments. In one embodiment, the dust samples can be collected within the structure and external to the structure. In some cases, the samples collected within the structure are collected from a top portion of a doorframe or another flat elevated surface within the structure.

In some cases, the machine learning estimator can include a Random Forest (RF) classifier. In some cases, the training can further include analyzing an internal transcribed spacer (ITS) region for each DNA sequence. In some cases, the training can further include identifying a set of Amplicon Sequence Variants (ASVs) for each collected sample from an individual structure. In some cases, the training can further include determining a primary taxonomic fungal grouping for each sample of the individual structure from the identified ASVs.

In some cases, at least one DNA sequence from the set of DNA sequences can be extracted from a mold sample collected from at least one mold-damaged structure.

In one embodiment, the steps of the computer-implemented method can be repeated. In some cases, the steps of the computer-implemented method can be repeated after the structure has been determined to have mold growth due to water damage.

In some cases, the structure can be determined to have mold growth due to water damage, and the structure, or a portion thereof, is removed from normal human use. In some cases, the structure can be determined to have mold growth due to water damage, and one or more mold remediation steps are carried out. In some cases, after the one or more mold remediation steps, the method can additionally include repeating the steps of the computer-implemented method.

In some cases, the remediation steps can be repeated until the structure is determined not to have mold growth due to water damage. In some cases, after the structure has been determined not to have mold growth due to water damage, the structure, or portion thereof, that has been removed from normal use by humans is returned to normal use by humans. In some cases, an accuracy for determining whether the structure has mold growth due to water damage is at least 90 percent.

In another aspect, a computer-readable medium includes a machine learning estimator trained to distinguish structures with mold growth due to water damage from structures without mold growth due to water damage.

In another aspect, a system for carrying out a computer-implemented method of identifying mold growth due to water damage in a structure is provided. The system can include an automated sample collector; a DNA sequencer; and a computer processor for determining by the machine learning estimator whether the structure has mold growth due to water damage.

This aspect can have a variety of embodiments. In some cases, the computer processor can be remote from the sample collector and DNA sequencer.

Another aspect of the disclosure provides a computer-implemented method of determining whether mold is present in a structure. The computer-implemented method includes: collecting a set of dust samples from the structure; extracting a set of DNA sequences from the set of dust samples; inputting the set of DNA sequences into a trained machine learning estimator; and determining by the machine learning estimator whether the structure experiences a predefined level of mold, a pattern of mold, a type of mold, or a combination thereof, based on the training.

This aspect can include a variety of embodiments. In some cases, an accuracy for determining whether the structure experiences the predefined level of mold, a pattern of mold, a type of mold, or a combination thereof, can be at least 90 percent.

Another aspect of the disclosure provides a computer-implemented method of identifying mold growth due to water damage in a structure. The computer-implemented method includes: receiving a first set of DNA sequences extracted from a set of dust samples collected from a plurality of mold-damaged structures; receiving a second set of DNA sequences extracted from a set of dust samples collected from a plurality of non-mold-damaged structures; and training a machine learning estimator using the first set of DNA sequences and the second set of DNA sequences, where the training includes at least: detecting differentially present DNA sequences for the first set of DNA sequences and the second set of DNA sequences; comparing a relative abundance of DNA sequences in the first set of DNA sequences and the second set of DNA sequences; and identifying from the detection and/or comparing at least one mycological difference between the set of dust samples from the plurality of mold-damaged structures and the set of dust samples from the plurality of non-mold-damaged structures.

Another aspect of the disclosure provides a computer-implemented method of identifying mold growth on building materials in a structure. The computer-implemented method includes: receiving a first set of DNA sequences extracted from a set of dust samples collected from a plurality of mold-damaged structures; receiving a second set of DNA sequences extracted from a set of dust samples collected from a plurality of non-mold-damaged structures; and training a machine learning estimator using the first set of DNA sequences and the second set of DNA sequences. The training includes at least: detecting differentially expressed genes for the first set of DNA sequences and the second set of DNA sequences; comparing a relative abundance of the first set of DNA sequences and the second set of DNA sequences from the differentially expressed genes; and identifying from the comparing at least one mycological difference between the set of dust samples for the plurality of mold-damaged structures and the set of dust samples for the plurality of non-mold-damaged structures.

This aspect of the disclosure can have a variety of embodiments. Dust samples collected for a mold-damaged structure or a non-mold-damaged structure can be collected within the structure and external to the structure. The samples collected within the structure can be collected from a top portion of a doorframe or another flat elevated surface within the structure.

The machine learning estimator can include a Random Forest (RF) classifier, Artificial Neural Networks classifier, linear regression classifier, logistic regression classifier, classification and regression trees (CART), Naïve Bayes classifier, K-Nearest Neighbors classifier, Apriori analysis, K-Means clustering, Principal Component Analysis (PCA), or Adaptive Boosting.

The training can further include analyzing an internal transcribed spacer (ITS) region for each DNA sequence.

The training can further include identifying a set of Amplicon Sequence Variants (ASVs) for each collected sample from an individual structure. The training can further include determining a primary taxonomic fungal grouping for each sample of the individual structure from the identified ASVs.

At least one DNA sequence from the first set of DNA sequences can be extracted from a mold sample collected from at least one of the mold-damaged structures.

Another aspect of the disclosure provides a computer-implemented method of determining whether mold is present in a structure. The computer-implemented method includes: collecting a set of dust samples from the structure; extracting a third set of DNA sequences from the set of dust samples; inputting the third set of DNA sequences into the machine learning estimator trained according to a method described herein; and determining by the machine learning estimator whether the structure experiences a predefined level of mold based on the training.

This aspect of the disclosure can have a variety of embodiments. An accuracy level for determining whether the structure experiences the predefined level of mold can be at least 90 percent.

### BRIEF DESCRIPTION OF THE DRAWINGS

For a fuller understanding of the nature of the present disclosure, reference is made to the following detailed description taken in conjunction with the accompanying drawing figures wherein like reference characters denote corresponding parts throughout the several views.

FIG. 1 depicts an exemplary method for identifying mold growth on building materials in a structure, according to an embodiment of the disclosure.

FIG. 2 depicts an exemplary method for determining whether mold is present in a structure, according to an embodiment of the disclosure.

FIG. 3 depicts a summary table of metadata collected for each dust or mold sample.

FIG. 4 depicts a list of differentially abundant taxa according to DESeq that were more abundant in (a) mold homes than the no-mold homes, and (b) no mold homes than mold homes.

FIG. 5 depicts a Köppen-Geiger climate classification map of the continental US overlaid with the locations of each home sampled. Each dot represents a home, with a darker grey indicating the overlay of multiple homes in a single city. In experimental study 1, temperate regions include Cfa, continental regions include Dfa and Dfb, and dry regions include Bsh, Bsk, and Bwh.

FIG. 6 depict charts of relative abundance of the top ASV of each sample: separated by (A) sample type and (B) the top 5 most abundant genera and sample type. Boxplots are overlaid with a scatterplot (25^{th} quartile, media, 75^{th} quartile) of the relative abundance for panel A.

FIG. 7 depicts a heatmap of the top 25 most abundant genera. Taxa were sorted by abundance with the most abundant on top. The heatmap is shaded by the log₁₀ read counts (a pseudocount of 0.1 added to each) with the most highly abundant taxa shaded lighter and those taxa that were not present in black. Samples are sorted into columns based on their sample type with bars across the top: direct mold, mold, no mold, and outside.

FIG. 8 depicts a chart of Log₂ fold changes in relative abundance for those taxa with a statistically significant fold change according to DESeq. Each point represents an individual ASV; therefore, some genera (e.g. Penicillium) have multiple points. ASVs are shaded according to their phylum annotation.

FIG. 9 depicts an NMDS plot of the Bray Curtis dissimilarities between all samples shaded by sample type. Statistically significant differences are apparent between the sample types (ANOSIM R=0.05, p=0.002). Samples are grouped by their sample type: direct mold—circles, mold—squares, no mold—diamonds, and outside—triangles.

FIG. 10 depicts NMDS plots of the Bray Curtis dissimilarities for each sample type shaded by Köppen Region. (A) Samples taken directly from the mold source (direct mold). (B) Samples from moldy homes (mold). (C) Samples from homes without mold damage (no mold). (C) Samples taken outside each home (outside). Axes are constrained so that x and y coordinates are equal within each plot. Samples from the continental region (Dfa, Dfb) are circles, samples from the dry region (Bsh, Bsk, Bwh) are squares, and samples from the temperate region (Cfa) are diamonds.

FIG. 11 depicts charts of Bray Curtis dissimilarities between samples. The panels of the figure compare the Bray Curtis dissimilarities for samples within a given house (yes: intra-home variability) and between different homes (no: inter-home variability) for a sample type. The median Bray Curtis dissimilarities are as follows: for direct mold, yes=0.72 and no=0.97; mold, yes=0.67 and no=0.95; and no mold, yes=0.79 and no=0.92. There is a statistically significant (p<0.001) difference in intra- and inter-home variability for each sample type.

FIG. 12 depicts a histogram of the number of correct predictions for each sample based on 20-fold cross-validation of the RF model.

FIG. 13 depicts a richness scatterplot of the number of ASVs. Boxplots (25^{th} quartile, median, 75^{th} quartile) are overlaid with each scatterplot. The 25^{th} quartile, median, and 75^{th} quartile are as follows for panel A: for direct mold 10, 16, and 26; mold: 11, 25, 70; no mold: 26, 55, 106; and outside: 26, 47, 88.

FIG. 14 depicts a heatmap of the statistically significant log₂ fold (log₂F) changes between mold and no mold house dust (p_{adj}≤0.01): (A) ASVs that were more abundant among the mold samples than the no mold samples and (B) ASVs that were more abundant among the no mold samples than the mold samples. Shaded bars on top of the figure represent the sample type. The heatmap is shaded by the log₁₀ relative abundances (0.1 added to each) with the most highly abundant taxa shaded lighter and those taxa not present in a sample in black.

FIG. 15 depicts NMDS plots of the Bray Curtis Dissimilarities between indoor dust samples in the no mold and mold homes: (A) all indoor dust samples, (B) all indoor dust samples using only differentially expressed ASVs, (C) indoor temperate samples, (D) indoor dry samples, and (E) indoor continental samples. Samples are shaded by their sample type: mold—squares and no-mold—diamonds.

FIG. 16 illustrates a comparison of the influence of direct mold and outside air on indoor fungal communities. Panel (A) provides a boxplot overlaid with a scatterplot of the number of ASVs in common for each indoor dust sample with the equivalent outdoor dust sample. There was a statistically significant difference between mold-outside and no mold-outside (Wilcoxon p<0.001). Panel (B) provides a boxplot overlaid with a scatterplot of the number of ASVs in common between direct mold and outside, as well as direct mold and mold for each house with mold damage. There was a statistically significant difference between direct mold and mold and direct mold and outside (Wilcoxon p=0.03). Panel (C) provides a scatter plot of the number of mold home air taxa also found in a direct mold sample from that home versus proximity to the nearest mold sample. The plot was overlaid with a line representing a linear model of the number of ASVs in common versus mold proximity. The equation of the line was found to be: y=−0.33*x+5.92. The influence of mold proximity on the number of taxa in common between mold and outside was statistically significant (adjusted R²=0.05, F(1,53)=3.84, p=0.055).

FIG. 17 depicts a chart of performance of the 20-fold cross-validated RF model on the subset of taxa that had a statistically significant change in abundance from mold to no mold. The circles overlaid with an X show the mean percent accuracy for the RF model using all of the samples.

FIG. 18 illustrates prediction accuracy for homes with three samples: (A) mold homes and (B) no-mold homes. Samples are shaded based on Köppen Region: Continental—circles, Dry—squares, and Temperate—diamonds.

FIG. 19 depicts a system for carrying out a computer-implemented method of identifying mold growth due to water damage in a structure, according to an embodiment of the disclosure.

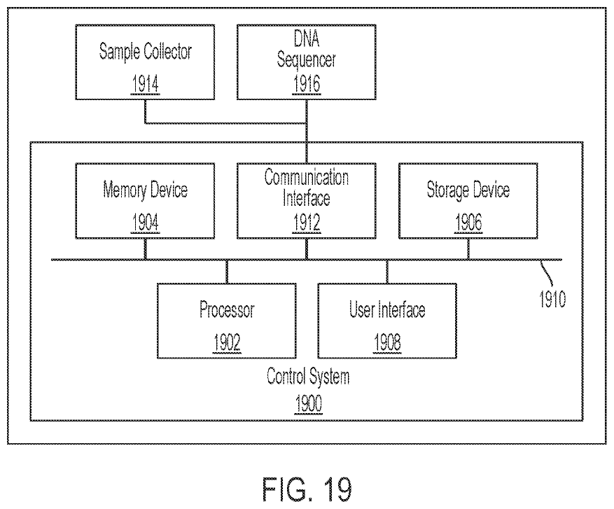

### DEFINITIONS

The instant disclosure is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Embodiments of the disclosure provide computer-implemented methods of identifying mold growth on building materials in a structure. The computer-implemented methods provide significant advantages over conventional techniques. For example, since the computer-implemented methods of the present disclosure are DNA-sequence-based (e.g., such as those provided in the corresponding Sequence Listing Table), they can produce ecological features such as richness as well as identify an essentially unlimited number of unique strains and species of mold.

Provided below are tables listing mold taxa that a conventional mold identification technique (the Environmental Relative Mold Index (ERMI)) can identify, as compared to mold taxa identifiable through the computer-implemented methods as disclosed herein. Table 1 lists taxa known to be associated with structures having water damage. In the left column of Table 1 are taxa identifiable by ERMI, whereas in the right column are taxa identified by an illustrative computer-implemented method of the present disclosure, the Mold Classification Tool (“MCT”). The MCT molds listed in Table 1 were identified in the working example below from homes with known mold infestations due to water damage. Because the MCT sequences samples rather than looking for a known set of molds, each use of it adds new sequences and allows it to improve with use. By contrast, the ERMI group is static.

TABLE 1
Mold Taxa Associated with Water Damage in a Structure

| ERMI group 1 | MCT mold |
|---|---|
| Aspergillus flavus | Preussia australis ASV 30 |
| Aspergillus fumigatus | Aspergillus niger ASV 1 |
| Aspergillus niger | Alternaria soliaridae ASV 7 |
| Aspergillus ochraceus | Cladosporium halotolerans ASV 8 |
| Aspergillus penicillioides | Aspergillus subversicolor ASV 67 |
| Aureobasidium pullulans | Penicillium sp. ASV 13 |
| Aspergillus restrictus | Penicillium sp. ASV 4 |
| Aspergillus sclerotiorum | Retroconis fusiformis ASV 22 |
| Aspergillus sydowii | Penicillium aurantiogriseum ASV 261 |
| Aspergillus unguis | Aspergillus piperis ASV 16 |
| Aspergillus versicolor | Stachybotrys echinate ASV 2 |
| Chaetomium globosum | |
| Cladosporium sphaerospermum | |
| Eurotium group | |
| Penicillium brevicompactum | |
| Penicillium corylophilum | |
| Penicillium group 2 | |
| Penicillium purpurogenum | |
| Penicillium spinulosum | |
| Penicillium variabile | |
| Paecilomyces variotii | |
| Scopulariopsis brevicaulis | |
| Scopulariopsis chartarum | |
| Stachybotrys chartarum | |
| Trichoderma viride | |
| Wallemia sebi | |

Table 2 lists mold taxa known to be associated with structures independent of water damage. In the left column of Table 1 are taxa identifiable by ERMI, whereas the right column are taxa identified in the working example below by a computer-implemented method of the present disclosure (MCT).

TABLE 2
Mold Taxa Associated with a Structure Independent of Water Damage

| ERMI group 2 | MCT no mold |
|---|---|
| Alternaria alternata | Malassezia restricta ASV 98 |
| Acremonium strictum | Pestalotiopsis sp. ASV 149 |
| Aspergillus ustus | Phoma crystallifera ASV 173 |
| Cladosporium cladosporioides Type 1 | Ustilago crameri ASV 221 |
| Cladosporium cladosporioides Type 2 | Phoma crystallifera ASV 68 |
| Cladosporium herbarum | Phoma crystallifera ASV 117 |
| Epicoccum nigrum | Neurospora terricola ASV 11 |
| Mucor group | Phanerochaete chrysorhiza ASV 227 |
| Penicillium chrysogenum Type 2 | Ustilago striiformis ASV 176 |
| Rhizopus stolonifera | Penicillium oxalicum ASV 29 |
| | Mucor racemosus ASV 46 |
| | Cladosporium sphaerospermum ASV 112 |
| | Mucor circinelloides ASV 77 |
| | Penicillium oxalicum ASV 32 |

Further, the computer-implemented methods are designed to continually collect training sets to improve classification accuracy and tool flexibility, which can include additional DNA sequences and additional sampled structures. The computer-implemented methods of the present disclosure also output more-advanced results relative to convention techniques. For example, the computer-implemented methods can not only detect mold levels of a sample, but also patterns of molds, confidence levels for a given classification, and other results classifying a sampled structure's fungal ecology. As is apparent from the working example below, “patterns of mold” can include differences in: the richness or diversity of the community (e.g., how many different taxa are present), the identifies of the taxa present, and the abundance of different taxa. An illustrative pattern of mold can include 168 individual molds identified and relative abundances between of all of them in the sample. Those of skill in the art readily appreciate that the method can be carried out without even identifying particular taxa, since the method is DNA sequence-based and can be carried out by identifying DNA sequences that are differentially present in samples from structures having mold growth due to water damage versus structures that do not have mold growth due to water damage.

### Exemplary Methods

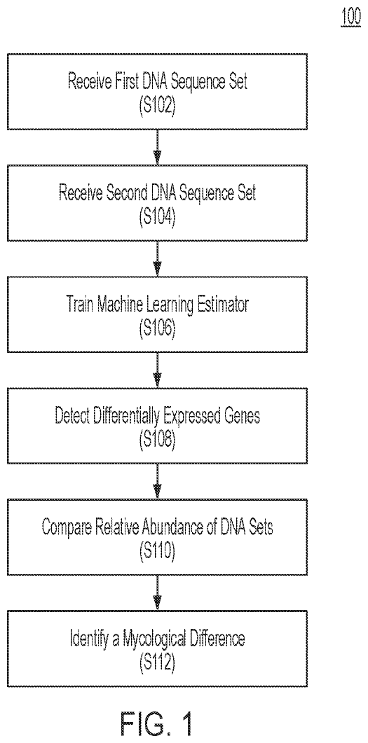

One exemplary method 100 for identifying mold growth on building materials in a structure is described and depicted in the context of FIG. 1.

In step S102, a first set of DNA sequences extracted from a set of dust samples can be received by a computer. The set of dust samples can be collected from a plurality of mold-damaged structures. For example, in some cases, the dust samples can be settled dust samples that have been uninterrupted for an extended period of time (e.g., at least 1 day, at least 1 week, at least 2 months, and the like). In some cases, the dust samples can be collected from elevated areas within the structure, such as from a doorframe, a window frame, a top rail of a door, a ceiling fan, the top of a piece of furniture, and the like. In some cases, the dust samples can be collected from the exterior of the structure, for example, within a predefined geographical range away from the structure (e.g., up to 30 meters away, etc.). In some cases, samples collected from the structure can also include samples taken from pieces of building materials with visible mold growth or water damage.

Further, embodiments of the disclosure can be implemented in whole or in part on a variety of computers including servers, personal computers, desktop computers, laptop computers, tablet computers, smartphones, and the like.

In step S104, a second set of DNA sequences extracted from another set of dust samples can be received by the computer. The set of dust samples can be collected from a plurality of non-mold-damaged structures, and can be collected similarly to the samples collected in step S102 (e.g., with regard to period since cleaning and location).

In step S106, a machine learning estimator can be trained using the first set of DNA sequences and the second set of DNA sequences. The machine learning estimator can be a part of the computer, or can be connected to the computer (e.g., hardwired, over a cloud network, via an intranet or internet, etc.). In some cases, the machine learning estimator can include a Random Forest classifier.

The training in step S106 can include a number of steps. For example, in step S108, the machine learning estimator can detect differentially expressed or differentially present genes for the first set of DNA sequences and the second set of DNA sequences. In particular, as shown in the working example below, the machine learning estimate can detect differences in RNA-encoding genes, or a portion thereof, such an internal transcribed spacer (ITS) region. An illustrative internal transcribed spacer (ITS) is the spacer DNA situated between the small-subunit ribosomal RNA (rRNA) and large-subunit rRNA genes in the chromosome or the corresponding transcribed region in the polycistronic rRNA precursor transcript. Those of skill in the art understand from the guidance presented herein that the method can be conveniently carried out by detecting any amplicon defined by primers to conserved genomic regions flanking a variable region and differentially present between samples.

The detection can occur through an RNA comparative analysis technique, such as DESeq2, and the like. In some cases, the machine learning estimator can identify a set of Amplicon Sequence Variants (ASVs) for each collected sample from a structure. The ASVs can be identified from analyzing an internal transcriber spacer (ITS) region for an extracted DNA sequence.

From the ASVs identified, the machine learning estimator can determine taxonomic fungal groupings of a collected sample. For example, the machine learning estimator can determine a primary taxonomic fungal grouping for a collected sample, or a set of taxonomic fungal groupings that dominate the collected sample.

In step S110, the machine learning estimator can compare a relative abundance of the first set of DNA sequences and the second set of DNA sequences from the differentially expressed or differentially present genes. The characteristics determined by the machine learning estimator, for example in step S106, can be compared between sets of DNA sequences. For example, the machine learning estimator can compare top ASV counts, a number of ASVs in a sample, a degree of ASV overlap between samples from the same structure (e.g., an outside sample and an inside sample, etc.), and the like.

In step S112, the machine learning estimator can identify, from the comparing, at least one mycological difference between the set of dust samples for the plurality of mold-damaged structures and the set of samples for the plurality of non-mold-damaged structures. In some cases, the machine learning estimator can identify a mycological pattern indicative of a mold-damaged structure or a non-mold-damaged structure. For example, peak ASVs, an ASV ratio within a sample, an ASV ratio between samples collected from the same structure, and the like, can be identified based on the comparison of DNA sequence sets.

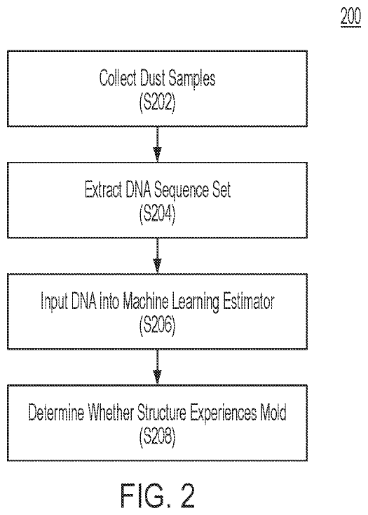

FIG. 2 depicts an exemplary method 200 for determining whether mold is present in a structure, according to an embodiment of the disclosure.

In step S202, a set of dust samples can be collected from the structure. The set of dust samples can be collected in various locations within and/or external to the structure. The set of dust samples can be collected from a plurality of non-mold-damaged structures, and can be collected similarly to the samples collected in step S102 (e.g., with regard to period since cleaning and location). In various embodiments, at least 3, 4, 5, 6, 7, 8, 9, 10 or more samples are collected.

In step S204, a set of DNA sequences can be extracted from the set of dust samples. In step S206, the extracted set of DNA sequences can be inputted into a machine learning estimator. In some cases, the machine learning estimator can have been previously trained according to the exemplary method 100 as described in more detail with reference to FIG. 1.

In step S208, the machine learning estimator can determine whether the structure experiences a predefined level of mold, a predefined type of mold, a predefined pattern of mold, e.g., characterized by the richness or diversity of the mold community (e.g., how many different taxa are present), the identities of the taxa and their relative abundance within a sample, or a combination thereof, based on the training. For example, the machine learning estimator can identify a set of characteristics of the set of dust samples collected from the structure, such as top ASV counts, a number of ASVs in a sample, a degree of ASV overlap between samples from the same structure, and the like. The machine learning estimator can then compare the identified characteristics with mycological differences found between mold-damaged structures and non-mold-damaged structures. In this way, the machine learning estimator can be used to determine whether the structure has mold growth due to water damage.

In some cases, an accuracy for determining whether the structure experiences the predefined level, type, or pattern of mold and/or whether the structure has mold growth due to water damage is at least 85, 86, 87, 88, 89, 90, 91, 92, 93, 94, 95, 96, 97, 98, or 99 percent.

Advantages of the claimed method, as compared to ERMI included the following. ERMI relies on 36 different targeted qPCR assays, 26 for group 1 (shown to be associated with homes with water damage), and 10 for group 2 (found in homes independent of water damage). By contrast, the methods described herein are DNA sequence-based. One sequencing run produces ecological features such as richness and potentially greater than 1,000 mold identifications depending on how many unique mold cells or spores are in a sample. For ERMI, results are presented in a score from −10 to +20, with higher numbers indicative of a greater presence of mold-based taxa from water damage. By contrast, the methods described herein, the machine learning output can classify a home's fungal ecology as moldy (e.g., having mold growth indicative of water damage) or normal. Based on model settings and the number of samples considered, confidence levels can be assigned to the classification. Accordingly the methods described herein provide a much more useful and actionable indication of the mold status of a structure. ERMI represents a static list of taxa that does not change for geography or as new information becomes available, such as the identity of molds that grow better on newer building materials not previously used. By contrast, the methods described herein are designed to continually collect training sets to improve classification accuracy and tool flexibility. Built into the approach is the idea that through application of the tool, additional homes and sequences can be added to the model training set. Accordingly, the method described herein can make use of information from taxa other than the static list of ERMI taxa.

Based on the results of method such as those described herein, appropriate steps can be taken to reduce adverse health consequences, such as removing a structure or portion of the structure from normal human use and optionally sealing it off, e.g., while one or more mold remediation steps are carried out.

Typically, mold remediation will involve cleaning up existing mold while avoiding exposure to the cleaner as well as homeowners or other occupants of the structure, as well as preventing new growth by addressing the moisture source. In some cases, for a contamination area of up to 30 square feet, guidelines provide for remediation levels 1 and 2. Level 1 remediation is used for small, isolated areas of mold up to 10 square feet, and Level 2 remediation covers square footage from 10 to 30 square feet.

Mold remediation protocols are well known and can include one or more of the following:

Repair the water problem. This will help prevent new mold spores from growing.

Isolate the contaminated area. Close all doors and windows between the contaminated area and other rooms of the home for both levels. For Level 2 remediation, also cover all doorways and any other openings with 6 mil polyethylene sheeting. Seal all seams of the sheeting with duct tape and slip openings in the sheeting to enter the contaminated area.

Suppress dust. Do this by misting the contaminated areas.

Remove materials. Remove all wet and mold-damaged porous materials. Consult Environmental Protection Agency (EPA) documents on mold remediation for information on which materials to remove.

Place materials in plastic bags. Discard all wet and moldy materials in plastic bags that are at least 6 mil thick, double-bag the materials, and tie the bags closed. The bags can be disposed of as regular trash once the outside of the bags are wiped with a damp cloth and detergent solution prior to leaving the contamination area.

Clean. All non-porous materials and wood surfaces that are moldy must be cleaned. Use a wire brush on all moldy surfaces and then wipe the area with disposable wipes. To dispose of as regular trash, discard wipes in 6 mil polyethylene bags, double-bag and tie closed. Finally, scrub all moldy surfaces using a damp cloth and detergent solution until all mold has been removed and rinsed cleaned surfaces with clean water.

Clean the affected area and egress. The process for Level 1 differs from Level 2 at this point. For Level 1, clean with a damp cloth and/or mop with detergent solution. Level 2 requires you to vacuum all surfaces with a HEPA vacuum, and then clean all surfaces with a damp cloth and/or mop and detergent solution. Discard wipes as described above.

Visibility test. All areas should be visibly free of contamination and debris.

Dry. Cleaned materials should be dried to allow leftover moisture to evaporate. To speed up the drying process, use fans, dehumidifiers or raise the indoor air temperature.

Replace. All materials that were moved should be replaced or repaired.

In various embodiments, retesting can be performed, e.g., to confirm a previous positive test or to determine the efficacy of one or more remediation steps. In some embodiments, retesting can be carried out until a method such as those described herein returns a result that the structure does not have water damage-related mold growth.

### System

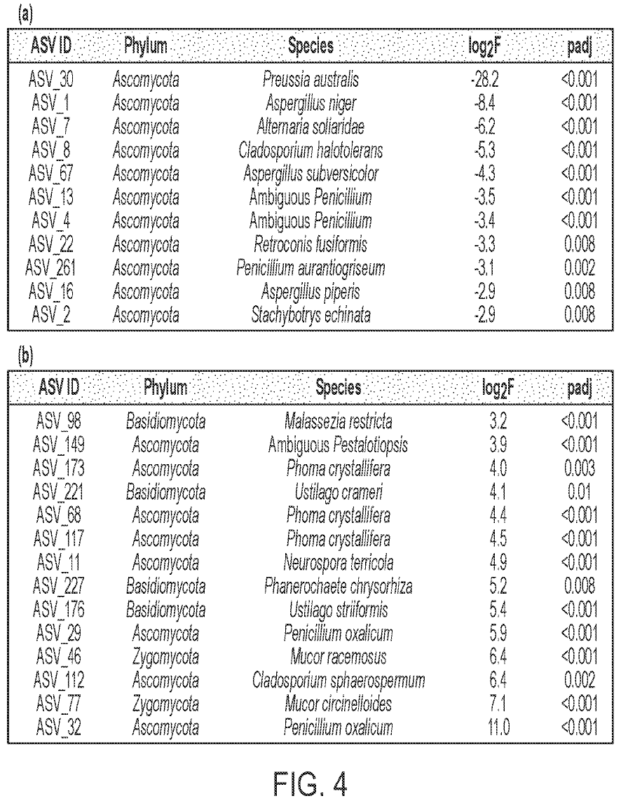

A system for carrying out the computer-implemented method of identifying mold growth due to water damage in a structure is depicted in FIG. 4. The control system 1900 can be a computing device such as a microcontroller (e.g., available under the ARDUINO® or IOIO™ trademarks), general purpose computer (e.g., a personal computer or PC), workstation, mainframe computer system, and so forth. The control system (“control unit”) 1900 can include a processor device (e.g., a central processing unit or “CPU”) 1902, a memory device 1904, a storage device 1906, a user interface 1908, a system bus 1910, and a communication interface 1912.

The processor 1902 can be any type of processing device for carrying out instructions, processing data, and so forth.

The memory device 1904 can be any type of memory device including any one or more of random access memory (“RAM”), read-only memory (“ROM”), Flash memory, Electrically Erasable Programmable Read Only Memory (“EEPROM”), and so forth.

The storage device 1906 can be any data storage device for reading/writing from/to any removable and/or integrated optical, magnetic, and/or optical-magneto storage medium, and the like (e.g., a hard disk, a compact disc-read-only memory “CD-ROM”, CD-ReWritable CDRW,” Digital Versatile Disc-ROM “DVD-ROM”, DVD-RW, and so forth). The storage device 1906 can also include a controller/interface for connecting to the system bus 1910. Thus, the memory device 1904 and the storage device 1906 are suitable for storing data as well as instructions for programmed processes for execution on the processor 1902.

The user interface 1908 can include a touch screen, control panel, keyboard, keypad, display or any other type of interface, which can be connected to the system bus 1910 through a corresponding input/output device interface/adapter.

The communication interface 1912 can be adapted and configured to communicate with any type of external device, such as the sample collector 1914 and the DNA sequencer 1916. The communication interface 1912 can further be adapted and configured to communicate with any system or network, such as one or more computing devices on a local area network (“LAN”), wide area network (“WAN”), wireless network (e.g., WiFi), low power, long-range wide area network (“LoRaWAN”), zigbee, Bluetooth, cellular, the Internet, and so forth. The communication interface 1912 can be connected directly to the system bus 1910 or can be connected through a suitable interface.

The control system 1900 can, thus, provide for executing processes, by itself and/or in cooperation with one or more additional devices or systems, that can include algorithms for carrying out a computer-implemented method of identifying mold growth due to water damage in a structure in accordance with the present disclosure. The control system 1900 can be programmed or instructed to perform these processes according to any communication protocol and/or programming language on any platform. Thus, the processes can be embodied in data as well as instructions stored in the memory device 1904 and/or storage device 1906, or received at the user interface 1908 and/or communication interface 1912 for execution on the processor 1902.

### WORKING EXAMPLE

### Experimental Study

Water damage in buildings and resultant mold growth is an ever-present public health issue. This study provides quantitative evidence for how the airborne fungal ecology of a damp building differs from the normal airborne fungal ecology of dry homes. A total of 288 indoor air (settled dust), outdoor air, and surface samples from building materials with direct mold were examined in 67 homes across dry, temperate, and continental climate regions within the continental United States. Community analysis based on the sequence of the Internal Transcribed Spacer (ITS) region of fungal ribosomal RNA encoding genes demonstrated consistent and distinct differences between the fungal ecology of settled dust in homes containing dampness and visible mold versus the settled dust of homes with no history of dampness or visible mold. These differences include lower community richness (p_{adj}=0.01) in the settled dust of damp homes versus dry homes, as well as distinct differences community taxonomic structure between damp and dry homes (ANOSIM R=0.15, p=0.001). A total of 11 taxa, all Ascomycota, were more highly enriched in damp homes while 14 taxa from Ascomycota, Basidiomycota and Zygomycota where more highly enriched in home with no history of mold or water damage. While climate region also exerted influence on fungal communities in all sample types (direct mold, indoor air, outdoor air), the differences between settled dust in damp versus dry homes were significant for all three climates considered. These distinct, but complex differences between mold and no mold sample used to train a Random Forest-based machine learning model to classify mold status of a home. The model was able to accurately classify 100% of mold homes and 90% of normal homes. This integration of DNA-based fungal ecology with advanced computational approaches can be used to accurately classify mold exposure in homes and assist with inspection and remediation industry and reduce hazardous microbial exposures indoors.

### Introduction

Building dampness caused by either a flooding event or high humidity is a common, global occurrence. Climate-driven shifts in regional and seasonal precipitation patterns and sea level rise are expected to compound this problem in many areas of the world. The presence of dampness, mold odors, or the resultant visible mold growth on building materials has been consistently associated with respiratory symptoms, the development of asthma, exacerbation of allergic disease, and neurological symptoms in building occupants.

Our understanding of the taxa or ecological characteristics associated with health impacts attributable to visible mold, odor, and dampness will be improved by identifying the fungal ecologies present in the air of dry buildings and damp buildings, and then revealing the differences between the two. These differences in ecology can then be leveraged to then reveal if a home has been impacted by hidden dampness-associated mold growth or if visible mold and dampness results in detectable airborne exposures that diverge from a building's normal fungal ecology. Such evidence is essential for home owners, public health researchers, mold inspectors, and building remediators when making decisions regarding the need for remediation or assessing post-remediation clearance.

Prior culture- and DNA-sequence-based studies on mold and dampness have set a useful baseline regarding the type of mold that grows on different building materials under variable water activities. Important examples of fungi growing on damp wood, drywall and ceramic materials include members of the genera Acremonium, Penicillium, Stachybotrys Ulocladium, Arthrinium, Aureobasidium, Aspergillus and Mucor. However, detection of these taxa in the air of buildings, where exposure occurs, does not reliably indicate the presence of dampness and mold. All of the above taxa naturally occur in the outdoor environment, and are thus transported into the indoor environment. Setting concentration thresholds for specific taxa or groups of taxa can be elusive due to the dearth of dose-response information as well as the temporally dynamic concentrations of fungi in building air that is caused by patterns of occupancy, human activity, and building operation. A final limitation is that databases or descriptions on what constitutes a normal fungal ecology have not been rigorously established. These must include natural fungal ecology at different climates and provide a full taxonomic resolution that is afforded by DNA sequencing approaches. Due to the large data sets produced on ecology and the potentially subtle differences that have been identified in fungal ecology between homes with visible mold and dry homes, the task of distinguishing between homes with and without mold damage is well-suited to machine learning algorithms, such as Random Forest (RF), Artificial Neural Networks, linear regression, logistic regression, classification and regression trees (CART), Naïve Bayes, K-Nearest Neighbors, Apriori analysis, K-Means clustering, Principal Component Analysis (PCA), or Adaptive Boosting. RF has been successfully applied to a diversity of classification problems in ecology.

The purpose of this study is to determine the differences in airborne fungal ecologies between the settled dust of inspector confirmed homes with reported moisture or visible mold versus homes with no known mold growth or moisture problems (normal ecology). This complex ecological information is then used classify homes as moldy or dry based on settled dust samples. A sampling campaign was conducted from diverse geographic and climatic regions throughout the US and included indoor air settle dust samples, as well as potential fungal sources including outdoor air and mold growing directly on building materials. High-throughput DNA sequencing of the internal transcribed spacer (ITS) region of the fungal rRNA-encoding gene and computational biology approach were utilized to identify quantitative ecological differences between the indoor air of dry versus damp homes from a variety of US climate regions. This study then utilizes the collected microbial community databases to develop and validate a machine learning approach that categorizes a home's fungal ecology as moldy or normal.

### Sampling Campaign

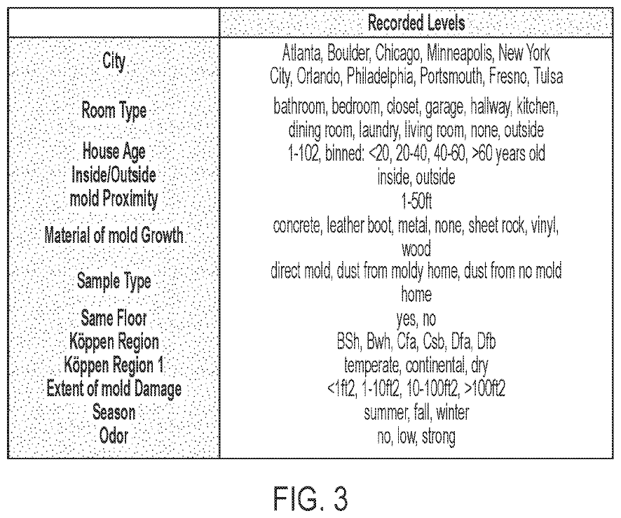

Samples were collected from diverse climatic regions across the U.S. by local, professional building inspectors and remediators. Eleven cities (Atlanta, Ga., Orlando, Fla., Tulsa, Okla., Denver, Colo., Phoenix, Ariz., Minneapolis, Minn., Portsmouth, N.H., Portland, Oreg., Chicago, Ill., Boulder, Colo., Philadelphia, Pa.) representing six climatic regions (humid subtropical (Cfa), humid continental (Dfa), temperate continental (Dfb), warm semi-arid (Bsh), warm desert (Bwh), and temperate Mediterranean (Csb)) were sampled. These climate regions were further binned into continental (Dfa, Dfb), temperate (Cfa, Csb), and dry climates (Bsh, Bwh). Typically up to 10 single family homes were selected in each city and included both homes with inspector-confirmed water damage and visible mold (“mold” samples), and homes with no history of water damage or visible mold (“no mold” samples). For each home, the following metadata was recorded: home age (binned: <20, 20-40, 40-60, >60 years old), home location (GPS or address converted to GPS), home size (floor area, m²), brief description of type of room or rooms sampled, distance (and floors) between sample and nearest direct mold (mold homes only), building material sampled from for direct mold samples (drywall, metal, ceramic, wood, other), observation of odor (strong, weak, none), current moisture condition of moldy building material (wet, damp, dry), area of direct mold (moldy homes, m²), and area of water damage (mold homes, m²) (FIG. 3).

Fungal samples were collected from surfaces using sterile cotton tipped swabs moistened with a filter sterilized 0.15 M NaCl, 0.1% TWEEN® solution. Two types of samples were taken and include settled dust samples from the tops of doorframes and surface swab samples from direct mold. Settled dust samples on elevated surfaces provide time-integrated air samples. For the “no mold” homes, up to three swab samples were collected from the tops of door frames within the home and one from an outside door frame. In “mold” homes, nine swab samples were collected in each home, including up to three indoor doorframe samples, one outdoor doorframe sample, and up to five samples directly from the surface of the material with mold growth (“direct mold”). The settled dust samples were collected from a 5 cm long portion of the upper doorframe and the thickness of the door frame was recorded. For direct mold, a 10 cm² section of the material was sampled. After sampling either the direct mold or settled dust, cotton swab tips were dropped into 2 mL screw top tubes and sent to the Yale University Environmental Biotechnology Lab for long-term storage at −80° C.

### Fungal Community Sequencing and Processing

The DNAEASY™ POWERSOIL® Kit (Qiagen Inc., Germantown, Md.) was used to extract DNA from the cotton swabs. In addition to the standard kit protocol, bead beating was used to improve cell lysis. In addition to lysing fungal cells, employing methods to lyse fungal spores, which are more resistant than cells to releasing nucleic acids, is an advantage because doing so increases the number of individual mold species and strains identified in the DNA sequencing step below. The increase in the number of individual mold species and strains from lysing spores produces richer signatures and provides the machine learning models described herein more information to work with and therefore a resulting increase in accuracy. Spore lysis is typically achieved through the use of enzymatic digestion with proteinase k, or with mechanical agitation employing variable sized glass beads or a combination of the two methods (see Fredricks D N, Smith C, Meier A. Comparison of six DNA extraction methods for recovery of fungal DNA as assessed by quantitative PCR. J Clin Microbiol. 2005; 43(10):5122-5128. doi:10.1128/JCM.43.10.5122-5128.2005).

The fungal internal transcribed spacer (ITS) region was amplified using the ITS-1F (CTTGGTCATTTAGAGGAAGTAA) and ITS2 (GCTGCGTTCTTCATCGATGC) primers. The University of Texas Genomic Sequencing and Analysis Facility (UT GSAF, Austin, Tex., USA) carried out library preparation, sequencing, and de-multiplexing of fungal sequences, generating 250 base-pair paired end reads.

Raw reads were downloaded and primers removed using CUTADAPT™ software. The reads were filtered and Amplicon Sequence Variants (ASVs, also known as Exact Sequence Variants) were created using the R software package DADA2. The use of ASVs offers improvements over operational taxonomic units (OTUs) through demonstrated improved sensitivity and specificity. Overall, 288 samples collected from 67 homes passed filtering and were utilized in this study. This included 58 outdoor samples, 59 indoor air samples from no mold homes, 58 indoor air samples from mold homes, and 113 direct mold samples. The standard DADA methodology was followed with the “pooling all samples strategy” and three mismatches were allowed in the alignment stage (mergePairs( . . . , maxMismatch=3). The ASV sequences were then BLASTed against the UNITE database and taxonomic identifications assigned using FHiTINGS™ software, version 1.4. Samples with fewer than 1000 reads were discarded.

### Fungal Community Analysis

To ascertain which sample types (direct mold, mold, no mold, and outside) had statistically significant differences in richness (ASV counts) and evenness (Shannon diversity), ANOVA (aov( ) stats v3.5.0) was used to test for a statistical difference in the alpha diversity measure, followed by Tukey's test (TukeyHSD( ) stats v3.5.0) to determine which sample types drove this difference. Bray Curtis dissimilarities were calculated based on the log₁₀ normalized relative abundances (pseudocount of 0.1) and then non-metric multidimensional scaling (NMDS) (ordinate( ) phyloseq v1.26.0) was used to visualize if the fungal communities clustered by type and region. ANOSIM™ (vegan v2.5-3) software was then used to test whether the observed differences in community were explained by region and sample type. NMDS analysis was repeated on only those taxa that were found to be differentially abundant by DESeq in mold and no mold homes.

To assess the degree of similarity among the samples, the Bray Curtis dissimilarities between samples of the same type in the same home and between samples of the same type, but in different homes were compared using the Welch two sample t-test (t.test( ) stats v3.4.0).

The Wilcoxon rank sum test with continuity correction (wilcox.test( ) stats v3.5.0) was used to test whether there was a statistically significant difference between the number of ASVs in common between both types of inside air and outside air, as well as between direct mold and mold and outside air. Additionally, a linear regression model (lm( ) stats v3.5.0) was used to determine whether the effect of direct mold on mold air changed with distance to the mold source.

A modified DESeq protocol was used to identify taxa that are differentially abundant between mold and no mold. DESeq was performed on the original (not rarefied) read counts after removing both those samples that had fewer than 1000 reads and those taxa that were not found in at least 20% of the samples. Additionally, in the calculation of size factors, the geometric mean was estimated based on those taxa that had a read count above zero for that sample rather than the standard method where the geometric mean was estimated for each sample based on only those that had no zero counts.

### Machine Learning Estimator Classification

An initial RF classifier was built using all fungal taxa that met our filtering criteria. Models were created using rfsrc( ) (randomForestSRC v2.8.0) with the default parameters and 20-fold cross-validation. For each iteration, seventy percent of the data was used for model development and thirty percent of the data was retained for validation. A second RF model using only those taxa that were found to be differentially abundant between mold and no mold was then built using the same process. This second model was used for all subsequent analyses.

Based on the mold and no mold sample comparative analyses above, sample richness, top taxa abundance, and influence of outdoor air on indoor air were all examined for differences between the samples that were commonly classified correctly and those that were commonly incorrectly classified. Richness in each sample was determined after rarefying to a depth of 1000 reads. The top ASV (Amplicon Sequence Variants) in each sample was the taxa with the highest relative abundance. The influence of outdoor air on the indoor air samples was measured as the number of ASVs in common between an indoor sample and the outdoor sample of that home. The Welch two sample t-test (t.test( ) stats v3.4.0) was used to determine whether these parameters had different patterns between the samples that the RF model correctly predicted in more than 15 of cross-validation iterations (“right >75%”) and those that it correctly predicted in fewer than 5 (“right <25%”).

Minimal depth was also varied and calculated for each of the 20 iterations of the RF model using gg_minimal_depth( ) (ggRandomForests v2.0.1). Minimal depth is the average of the depth of the first split of each variable across all trees; the quantitative threshold for variable importance used in this study regards all variables with a minimal depth below the mean minimal depth as important for classification. Any taxa with a minimal depth under this threshold in at least 19 of the 20 RF iterations was considered an important taxa for this study.

### Overview of Samples

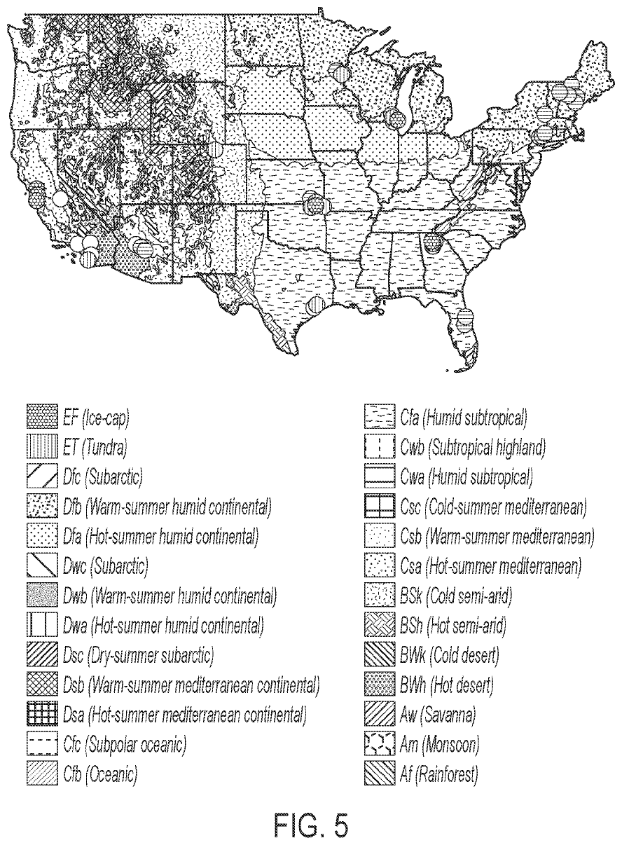

Homes across six Köppen climate zones (Cfa, Dfa, Dfb, Bsh, Bsk, and Bwh) were sampled. For geographic-based analyses, the regions were grouped into temperate (Cfa), continental (Dfa and Dfb), and dry (Bsh, Bsk, and Bwh) regions. Temperate, continental, and dry climates represent the majority of the continental U.S. by land area and population, excluding some alpine zones in the Rocky Mountains and a tropical zone at the tip of Florida. Four categories of samples were collected, including: (1) indoor settled dust from homes with no history of water damage or visible mold (“no mold” home dust), (2) indoor settled dust from homes with inspector documented water damage and visible mold (“mold”), (3) outdoor settled dust from all homes (“outside”), and (4) direct surface samples from building materials (e.g. wood, ceramic, drywall) with visible fungal growth (“direct mold”). See FIG. 3 for home descriptions and FIG. 5 for a map illustrating the sampling campaign.

### Richness and Dominant Taxa

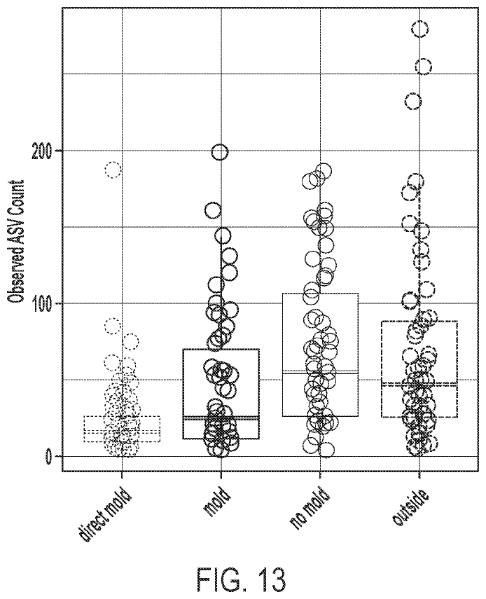

We observed differences in α-diversity between the sample types, with “direct mold” and “mold” settled dust samples having a lower richness than “no mold” and “outdoor air” settled dust. Statistically significant differences in the median richness levels (Tukey's test, p_{adj}<0.001) were found between the settled dust samples (outside, no mold, and mold samples) and the direct mold samples (FIG. 13). Within the settled dust samples, median richness in the mold samples was lower than the no mold and outdoor air samples (Tukey's test, p_{adj}<0.05).

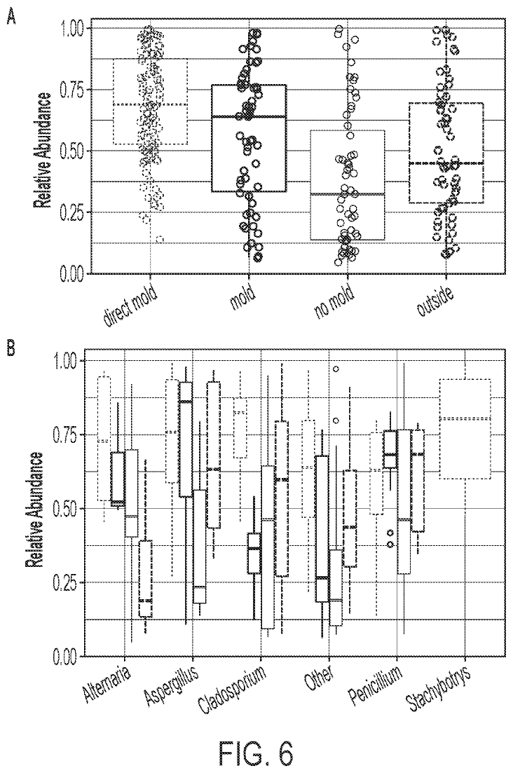

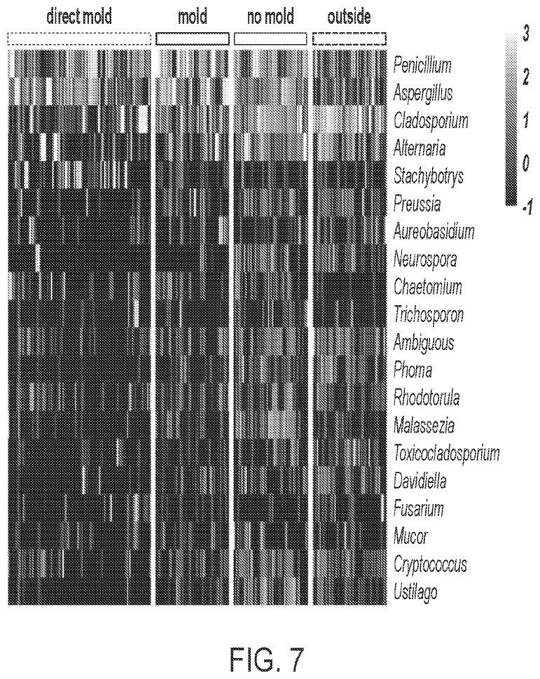

Direct mold samples tend to be dominated by a single, highly abundant taxa (ASV) (FIG. 6). The median relative abundance of the top ranked ASV is 69% for direct mold and 64% for mold, dropping to 45% for outside and 32% for no mold. A total of 54 different genera were the top taxa in at least one sample. Across all sample types, Aspergillus, Cladosporium, Penicillium, Alternaria, and Stachybotrys are the five most common top ASV genus annotations (FIG. 6) and the five most common genus annotations overall (FIG. 7). Within a sample, an average of 32% of the direct mold reads, 35% of mold reads, 14% of no mold reads, and 20% of outside reads are represented by these five genera. The proportion of reads annotated as these five genera was lower in no mold than mold (Tukey's test, p_{adj}=0.006 and p_{adj}=0.002, respectively).

Stachybotrys was the only top ASV in only one sample type (direct mold). Aspergillus and Penicillium were more commonly (˜2 times) the top ASV in mold compared to no mold. The difference in relative abundance between mold and no mold is statistically significant for the samples where Aspergillus was the top ASV (Tukey's test, p_{adj}=0.05), but not when Penicillium was the top ASV (Tukey's test, p_{adj}=0.31).

Taxa that Drive the Differences Between “Mold” and “No Mold” Air Samples

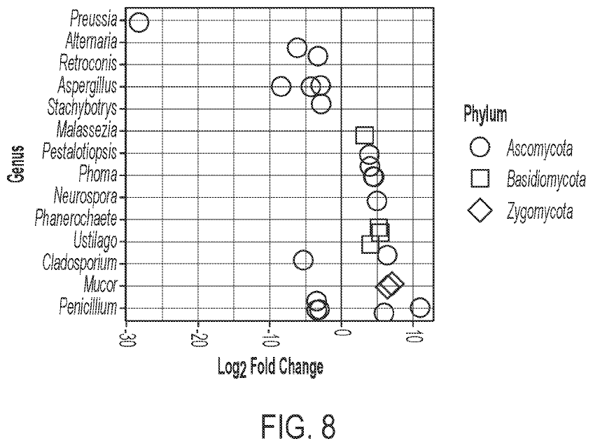

A quantitative comparative analysis revealed several ASVs that were differentially enriched between “no mold” and “mold” settled dust (FIGS. 4, 8, and 14). A total of 11 ASVs, all Ascomycota, were statistically overabundant in the mold homes, while 14 distinct ASV, covering Ascomycota, Basidiomycota, and Zygomycota, were statistically overabundant in no mold homes. The genera Aspergillus, Penicillium and Cladosporium have ASVs that are differentially expressed in opposite directions (FIG. 8). For Penicillium, ASVs 4 and 13 (ambiguous at the species level) and ASV 261 (top BLSAST hit Penicillium aurantiogriseum) were found to be more common in “mold” than “no mold”, with relative abundance (“mold”/“no mold”) log 2 fold changes (log 2F) of 3.4, 3.5, and 3.1 respectively. ASVs 29 and 32 (Penicillium oxalicum top BLAST hit for both) were found to be more common in “no mold” than in “mold”, with log 2FF changes of −5.9 and −11.0, respectively (mold/no mold). For Cladosporium, ASV 8 (top BLAST hit Cladosporium halotolerans) was more highly abundant in “mold” than “no mold”, with a 5.3 log 2F change, while ASV 112 (top BLAST hit Cladosporium sphaerospermum), was more abundant in the “no mold” than the “mold” samples: −6.4 log 2F change. For Aspergillus: ASVs 1, 16, and 67 (top BLAST hits Aspergillus niger, Aspergillus piperis, and Aspergillus subversicolor, respectively) were found to be more common in “mold” than “no mold”, with relative abundance (“mold”/“no mold”) log 2F changes of 8.4, 2.9, and 4.3, respectively.

### Fungal Communities Cluster by Sample Type

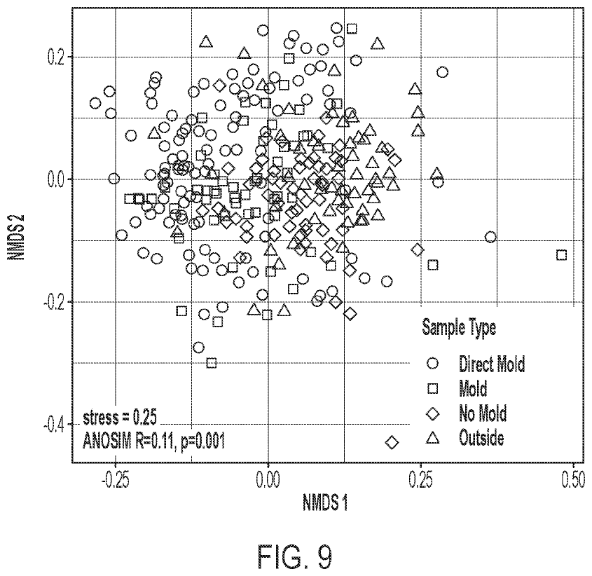

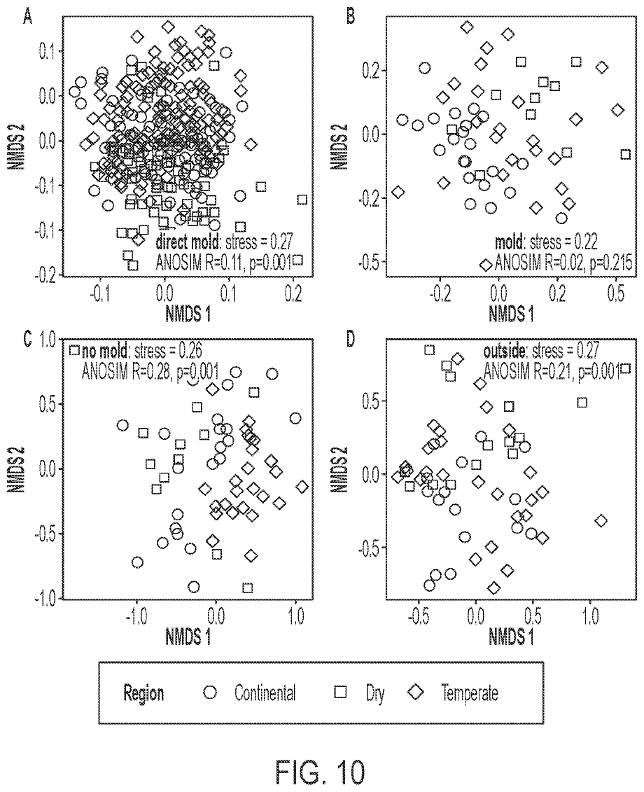

Ordination plots based on the Bray Curtis dissimilarities demonstrate differences in fungal community composition the sample types (ANOSIM R=0.11, p=0.001) (FIG. 9). “No mold”, “outdoor”, and “direct mold” samples also demonstrated distinct differences based on Köppen Region (dry—Bsh, Bsk, Bwh, temperate—Cfa, and continental—Dfa, Dfb) (ANOSIM p≤0.001); however, mold samples did not (ANOSIM p=0.215) (FIG. 10).

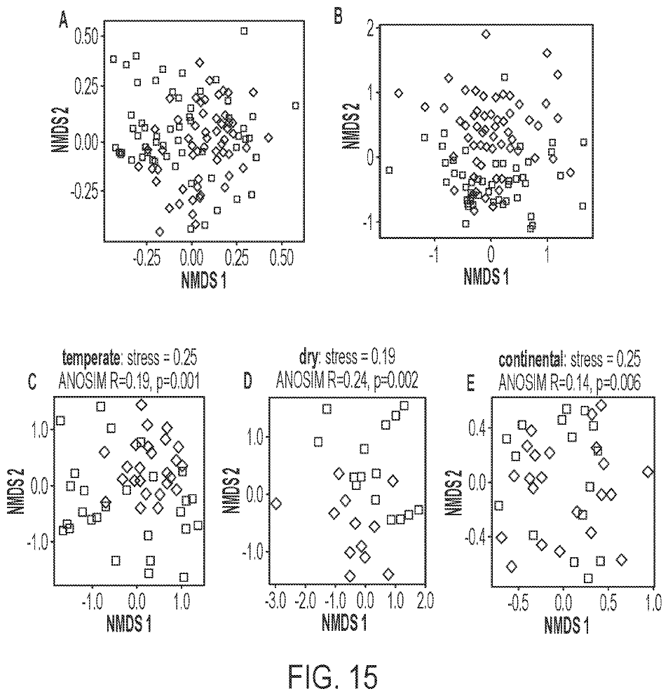

Additional Bray Curtis dissimilarity ordination plots (FIG. 15) revealed community differences between “mold” and “no mold” homes (ANOSIM R=0.15, p=0.001). Bray Curtis dissimilarity-based comparisons between “mold” and “no mold” using only those taxa that were differentially abundant between “mold” and “no mold” (FIGS. 3 and 4) improved the clustering (FIG. 15) (ANOSIM R=0.17, p=0.001). When separating by Köppen climate region, distinction between “mold” and “no mold” communities retained their statistical significance (ANOSIM p<0.01) for each region and suggest that climate does not drive “mold” versus “no mold” differences (FIG. 15).

### Intra-Home Comparisons

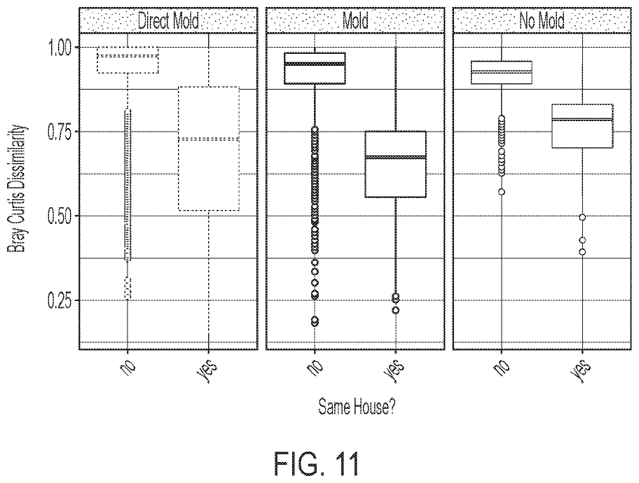

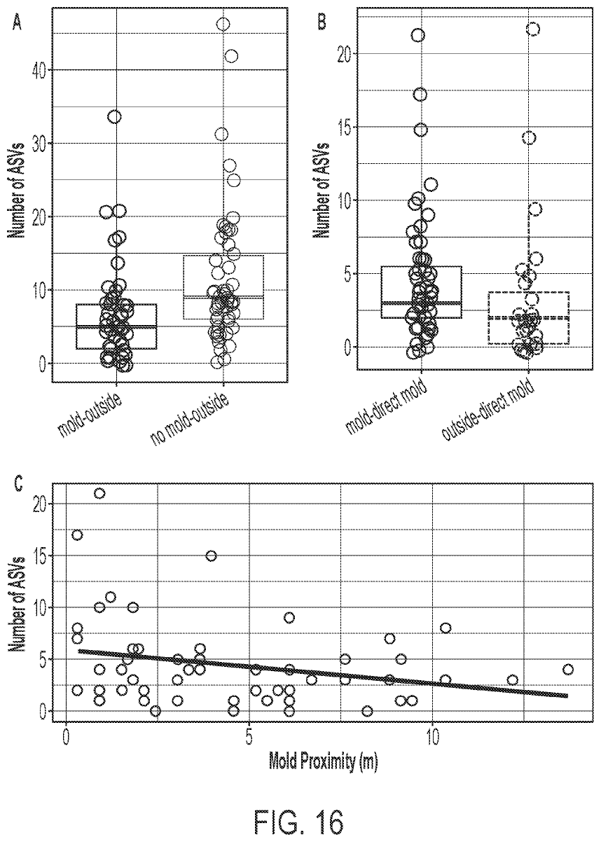

A final quantitative approach for comparing fungal ecologies between “mold” and “no mold” homes is through intra-home comparisons, which controls for climate and the many home-specific factors such as occupation, construction type, cleaning, and ventilation. The indoor dust ecology of moldy homes is expected to be influenced by both direct mold and fungi from outdoor air, while the indoor dust of no mold homes should have no influence from direct mold taxa. FIG. 16 demonstrates that the number of ASVs in common between a given inside sample and that home's outside sample is higher for no mold compared to mold homes (Wilcoxon rank sum test, p<0.001). Within “mold” homes, there are more ASVs in common between mold and direct mold than between outside air and direct mold (Wilcoxon rank sum test, p=0.03). A linear regression model of the number of ASVs in common between direct mold and mold samples versus proximity of a mold sample to direct mold reveals that the effect of direct mold on settled dust decreases slightly with distance from mold damage (R²=0.05; F(1,53)=3.84; p=0.055). Finally, multiple “direct mold”, “mold” and “no mold” samples were taken for each home, allowing for intra-versus inter-home comparisons of variability. Based on Bray Curtis dissimilarities between samples, variability between similar sample types from the same home (intra-home) is statistically less (t-test, p<0.001) than the variability between similar samples types in different homes (inter-home) (FIG. 11).

### Machine Learning Estimator Development and Optimization

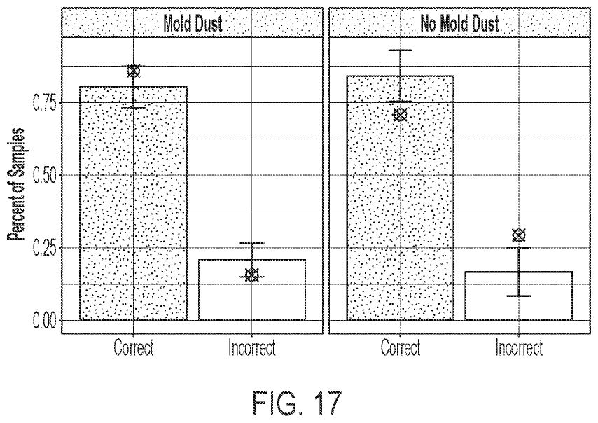

The above differences in “mold” versus “no mold” samples can be exploited through a machine learning approach to classify homes a “mold” (moldy ecology) or “no mold” (normal ecology). An initial cross-validated Random Forest model built using all taxa had an average accuracy of 82±10% across all samples. Most of this error comes from misclassifying “no mold” samples as “mold”; the average accuracy for the “mold” samples is 86±8%, while 70±12% of the “no mold” samples are correctly classified. Using only the differentially abundant taxa in the machine learning training sets also improves the accuracy of the RF model constructed (FIG. 17). The 20-fold cross-validated model's accuracy improves to an average of 83±9% for the “no mold” dust when built using only the differentially abundant taxa and remains roughly equivalent for the model as a whole (mean accuracy across all samples: 81±9%).

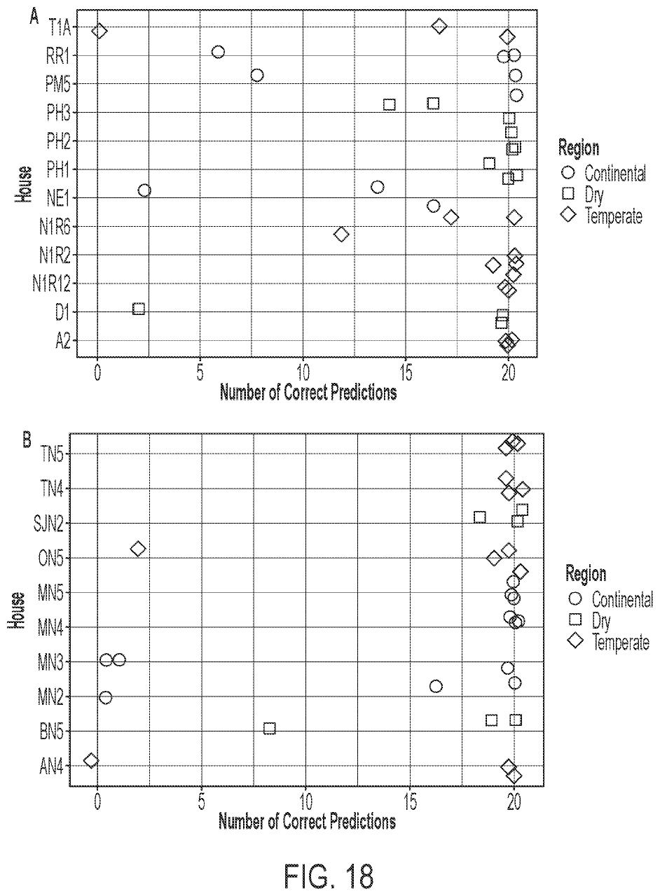

For those homes with at least 3 samples (22 homes), 21 are correctly classified as moldy or not moldy using the RF model based on only the differentially abundant taxa when using the classification of at least two out of the three samples. All (12 of 12) of the moldy homes were correctly identified in more than 50% of the RF model iterations when using this benchmark (FIG. 18), while 90% (9 or 10) of the no mold homes were accurately classified using the same. Focusing on the “no mold” home (Minneapolis-3) that was misclassified in more than 50% of the RF models for two of the three samples in that home, the misclassified samples have higher abundance of ASVs (e.g. ASV 4 and ASV 13) that were found to be more highly abundant in the “mold” homes (FIG. 4(a)) than of any ASVs associated with “no mold” (FIG. 4(b)).

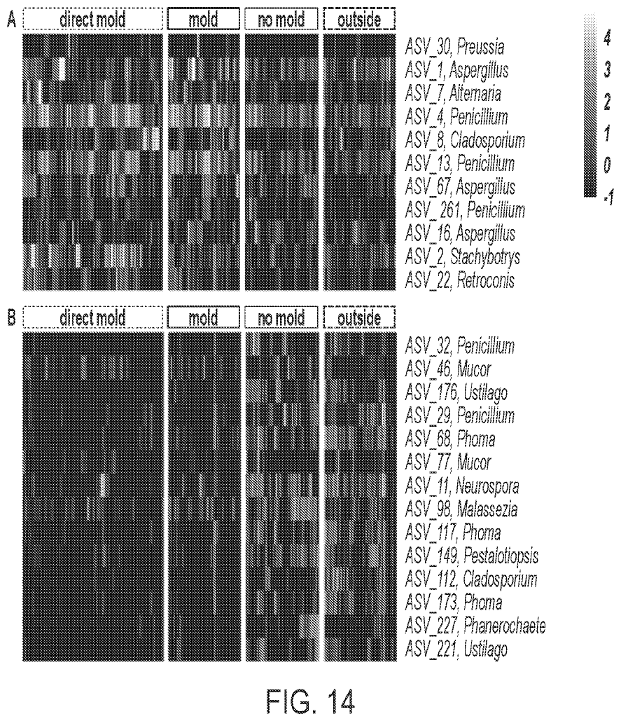

Minimal depth of variables was used to select the most important taxa for the RF model built using the differentially abundant ASVs (FIG. 14). Eight ASVs were deemed important in sufficient model iterations: Pestalotiopsis (ASV 149), Neurospora (ASV 11), Penicillium (ASV 29), and Malassezia (ASV 98) which were more abundant in “no mold” than “mold” and Aspergillus (ASV 1), Penicillium (ASVs 13 and 4), and Cladosporium (ASV 8) which were more abundant in “mold” than “no mold”.

### Machine Learning Estimator Prediction Accuracy

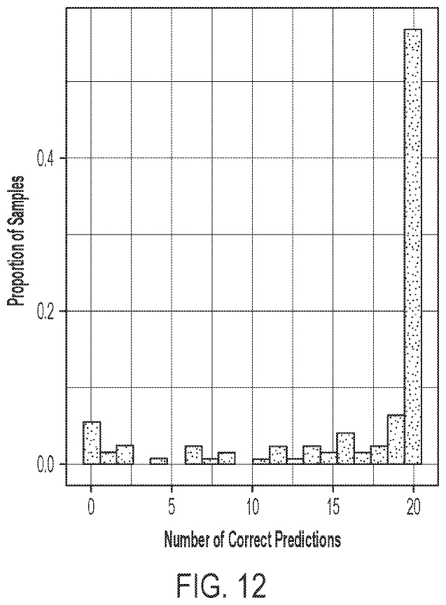

The RF model predicts most samples (60%) correctly in each iteration (FIG. 12). However, opposite patterns for top ASV count, number of ASVs in a sample, and degree of overlap with outside settled dust, influence correct classification. For the samples accurately classified in more than 75% of the cross-validation models, the relative abundance of the top ASV (top ASV count) was higher in mold than no mold (t-test, p=0.001). Conversely, a higher, but not statistically significant (t-test, p=0.12) top ASV count was observed in “no mold” than “mold” for the samples that the RF model predicts accurately less than 25% of the time. Richness was higher in the “no mold” versus “mold” for the samples that the RF model predicts correctly more than 75% of the time (t-test, p<0.001), while no difference was detected for the samples that the RF model classifies accurately less than 25% of the time (t-test, p=0.68). Regarding overlap with outdoor air, a higher number of ASVs are in common between the inside dust sample and that sample's outside sample in the “no mold” than “mold” samples for the samples that the RF model predicts correctly more than 75% of the time (t-test, p<0.001), mold samples are equally similar to outside ones for those samples that the RF accurately classifies less than 25% of the time (t-test, p=0.66). Each of these trends reflects observations from the comparative DNA sequence analysis.

### Discussion

This study uniquely integrated DNA sequence-based ecological approaches with modern computational biology and a multi-climate zone, in-depth building sample design to determine the ecological differences between fungi in the air of damp versus dry homes. Differences in ecology assessed by richness, patterns of top ASV enrichment, and differential taxa were quantified and leveraged to train a machine-learning model that classifies a home's airborne fungal exposure as moldy or dry (normal fungal ecology) with 95% accuracy. The findings of this study are novel. We are not aware of prior published studies that have revealed tangible DNA-based fungal ecology differences in homes with and without inspector-confirmed mold damage over multiple climate zones. This work represents a potentially significant advance in identifying and preventing human health impacts from damp buildings for two specific reasons: First, visible mold and water damage is associated with negative human health effects. By comparing the ecology in inspector-confirmed homes with and without visible mold, the microbial community signature that is ascribed to homes with visible mold is described. Second, the tools developed can be practically applied by mold inspectors and the remediation industry for guidance to determine if the fungal exposures in a building are associated with dampness, and to assess if a building has been cleared of these exposures due to remediation.

### Unique Fungal Community Characteristics Ascribed to Damp/Moldy Homes

“Mold” and “direct mold” samples had a lower richness than “no mold” samples. These patterns are consistent with recent studies on single homes, and damp building materials that suggest direct mold presence depresses the richness of fungi occupants are exposed to. “Direct mold” had significantly lower richness than indoor air or outdoor air in this study, and was dominated by few taxa. Prior studies have noted that the presence of a distinct fungal source with low richness can result in a reduced measured richness in indoor air. An important public health consequence is exposure to low fungal and bacterial richness in early life has been empirically associated with asthma development. The presence of visible mold, dampness, and mold odors, has also been associated with asthma development. Thus, the reduction in fungal richness in damp homes with visible mold is consistent in direction with this health impact.

Taxa responsible for differences in “mold” and “no mold” communities were estimated by comparative analysis. The dominant taxa in “mold” samples, belonged mostly to genera that have commonly been identified in prior culture-based studies and include Aspergillus, Penicillium, Stachybotrys, Cladosporium, and Alternaria. ASV-level analysis allowed for deeper insights: the three most common genera (Aspergillus, Penicillium, and Cladosporium) were highly abundant in all sample types, but specific ASVs were differentially abundant between “mold” and “no mold” samples. Many of the taxa highly enriched in “mold” homes, (all Ascomycota) have demonstrated public health significance; they are known allergens, produce mycotoxins, and (in the case of Penicillium) have been implicated as important for asthma development. The taxa highly abundant in “no mold” homes included members of Ascomycota, Basidiomycota, and Zygomycota. Many of the ASV's that are more highly abundant in “no mold” versus “mold” include taxa that are commonly found on human skin (Malassezia) or that are known to be common in the outdoor environment (eg. Phoma, Cladosporium, and Pestalotiopsis).

Beta diversity analysis demonstrated fungal community membership differences between “mold” and “no mold” homes, strong differences between “direct mold” and “outdoor air”, and the importance of considering climate zone. Climate (temperate, dry, and continental) appeared to impact fungal ecology not only for outdoor air, but for indoor air in homes without mold damage, and even direct mold. There is precedent for observing different fungal communities in buildings and outdoor air based on geography, largely through the association of fungi with outdoor plants as endophytes, micorrhizae, pathogens, or saprophytes. The community differences between “mold” and “no mold” homes were consistent, even when accounting for the three climate regions considered.

### Tools for Classifying Homes as Moldy or “Normal”

Building inspectors often utilize the abundance of Aspergillus or Pen/Asp to assess whether a home needs remediation. These taxa grow in damp buildings, have known allergic impacts on humans, and can be identified via culture or direct microscopy. However, neither the Aspergillus (t-test, p=0.98) nor Penicillium genera (t-test, p=0.42) abundances were different between the “mold” and “no mold” homes surveyed here. While Aspergillus and Penicillium genera abundances are indistinguishable between “mold” and “no mold” homes, both have ASVs that are differentially abundant between “mold” and “no mold” homes. The RF model constructed here illustrates the benefit of considering multiple taxa simultaneously and the necessity of moving beyond genera level identification. The accuracy of the RF model is enabled through considering the cumulative effect of numerous taxa (typically species level classification) associated with “mold” but also with “no mold” homes altogether. Determining which taxa are most important for classification can reveal further insights about the mycology of homes with and without mold damage.

Recent DNA-barcoding studies have revealed that the fungal diversity of the built environment is more complicated than previously accounted for by prior culture approaches. RF is a common, highly robust machine learning strategy particularly well-suited to classification problems in ecology. The development of sequence-based tools leveraging recent advances in machine learning is ideal for the classification of moldy and non-moldy homes. It further demonstrates, for the first time, the feasibility of using machine learning to classify homes by their mold status. The use of multiple predictions per sample, multiple samples per home, and a selected group of taxa allowed for independently and correctly identifying 100% of “mold” homes and 90% of “no mold” homes. While RF can be applied to a large number of taxa and other sample characteristics simultaneously, selecting those parameters most critical for classification is often required for model optimization. The machine learning model developed here illustrates the importance of considering a diverse set of fungi beyond simply the presence of common mold associated taxa, such as Aspergillus, as well as the benefit of limiting selection to only parameters (taxa in this case) important for the particular classification problem.

### EQUIVALENTS

Although preferred embodiments of the disclosure have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of identifying mold growth due to water damage in a structure, the computer-implemented method comprising:
receiving a set of DNA sequences extracted from one or more dust samples collected from the structure;
analyzing the sequences using a machine learning estimator, wherein the machine learning estimator has been trained to distinguish structures with mold growth due to water damage from structures without mold growth due to water damage; and
determining if the structure has mold growth due to water damage.

2. The computer-implemented method of claim 1, wherein dust samples collected for a mold-damaged structure or a non-mold-damaged structure are collected within the structure and external to the structure.

3. The computer-implemented method of claim 2, wherein the samples collected within the structure are collected from a top portion of a doorframe or another flat elevated surface within the structure.

4. The computer-implemented method of claim 1, wherein the machine learning estimator comprises a Random Forest (RF) classifier.

5. The computer-implemented method of claim 1, wherein the training further comprises analyzing an internal transcribed spacer (ITS) region for each DNA sequence.

6. The computer-implemented method of claim 1, wherein the training further comprises:
identifying a set of Amplicon Sequence Variants (ASVs) for each collected sample from an individual structure.

7. The computer-implemented method of claim 6, wherein the training further comprises:
determining a primary taxonomic fungal grouping for each sample of the individual structure from the identified ASVs.

8. (canceled)

9. (canceled)

10. The computer-implemented method of claim 1, additionally comprising repeating the steps of claim 1.

11. The computer-implemented method of claim 10, wherein the steps of claim 1 are repeated after the structure has been determined to have mold growth due to water damage.

12. The computer-implemented method of claim 1, wherein the structure is determined to have mold growth due to water damage, and the structure, or a portion thereof, is removed from normal human use.

13. The computer-implemented method of claim 1, wherein the structure is determined to have mold growth due to water damage, and one or more mold remediation steps are carried out.

14. The computer-implemented method of claim 13, wherein after the one or more mold remediation steps, the method additionally comprises repeating the steps of claim 1.

15. The computer-implemented method of claim 14, wherein the steps of claim 1, followed by remediation are repeated until the structure is determined not to have mold growth due to water damage.

16. The computer-implemented method of claim 15, wherein after the structure has been determined not to have mold growth due to water damage, the structure, or portion thereof, that has been removed from normal use by humans is returned to normal use by humans.

17. (canceled)

18. A computer-readable medium comprising a machine learning estimator trained to distinguish structures with mold growth due to water damage from structures without mold growth due to water damage.

19. A system for carrying out the computer-implemented method of identifying mold growth due to water damage in a structure according to claim 1, wherein the system comprises:
an automated sample collector;
a DNA sequencer; and
a computer processor for determining by the machine learning estimator whether the structure has mold growth due to water damage.

20. (canceled)

21. A computer-implemented method of determining whether mold is present in a structure, comprising:
collecting a set of dust samples from the structure;
extracting a set of DNA sequences from the set of dust samples;
inputting the set of DNA sequences into a trained machine learning estimator; and
determining by the machine learning estimator whether the structure experiences a predefined level of mold, a pattern of mold, a type of mold, or a combination thereof, based on the training.

22. (canceled)

23. A computer-implemented method of identifying mold growth due to water damage in a structure, the computer-implemented method comprising:
receiving a first set of DNA sequences extracted from a set of dust samples collected from a plurality of mold-damaged structures;
receiving a second set of DNA sequences extracted from a set of dust samples collected from a plurality of non-mold-damaged structures; and
training a machine learning estimator using the first set of DNA sequences and the second set of DNA sequences, wherein the training comprises at least:
detecting differentially present DNA sequences for the first set of DNA sequences and the second set of DNA sequences;
comparing a relative abundance of DNA sequences in the first set of DNA sequences and the second set of DNA sequences; and
identifying from the detection and/or comparing at least one mycological difference between the set of dust samples from the plurality of mold-damaged structures and the set of dust samples from the plurality of non-mold-damaged structures.

24. A computer-implemented method of identifying mold growth on building materials in a structure, the computer-implemented method comprising:
receiving a first set of DNA sequences extracted from a set of dust samples collected from a plurality of mold-damaged structures;
receiving a second set of DNA sequences extracted from a set of dust samples collected from a plurality of non-mold-damaged structures; and
training a machine learning estimator using the first set of DNA sequences and the second set of DNA sequences, wherein the training comprises at least:
detecting differentially expressed genes for the first set of DNA sequences and the second set of DNA sequences;
comparing a relative abundance of the first set of DNA sequences and the second set of DNA sequences from the differentially expressed genes; and
identifying from the comparing at least one mycological difference between the set of dust samples for the plurality of mold-damaged structures and the set of dust samples for the plurality of non-mold-damaged structures.

25.-31. (canceled)

32. A computer-implemented method of determining whether mold is present in a structure, comprising:
collecting a set of dust samples from the structure;
extracting a third set of DNA sequences from the set of dust samples;
inputting the third set of DNA sequences into the machine learning estimator trained according to the method of claim 24; and
determining by the machine learning estimator whether the structure experiences a predefined level of mold based on the training.

33. (canceled)
